(19) United States
(12) Patent Application Publication
(10) Pub. No.: US 2022/0386944 A1
(43) Pub. Date: Dec. 8, 2022

(54) SLEEP STAGING USING MACHINE LEARNING

(71) Applicant: Apple Inc., Cupertino, CA (US)

(72) Inventors: Alexander M. Chan, San Carlos, CA (US); Nader E. Bagherzadeh, Santa Clara, CA (US); Matt T. Bianchi, Santa Cruz, CA (US)

(21) Appl. No.: 17/339,894

(22) Filed: Jun. 4, 2021

(51) Int. Cl. A61B 5/00; G06N 20/00; G16H 40/67; G16H 50/20; G16H 50/30; G16H 50/70; A61B 5/08; A61B 5/11

(52) U.S. Cl. CPC A61B 5/4809; G06N 20/00; G16H 40/67; G16H 50/20; G16H 50/30; G16H 50/70; A61B 5/0816; A61B 5/1118; A61B 5/7264; A61B 5/7275; A61B 5/7203

(57) ABSTRACT

Embodiments are disclosed for sleep staging using machine learning. In an embodiment, a method comprises: receiving, with at least one processor, sensor signals from a sensor, the sensor signals including at least motion signals and respiratory signals of a user; extracting, with the at least one processor, features from the sensor signals; predicting, with a machine learning classifier, that the user is asleep or awake based on the features; and computing, with the at least one processor, a sleep or wake metric based on whether the user is predicted to be asleep or awake.

### TECHNICAL FIELD

This disclosure relates generally to sleep/wake tracking and machine learning.

### BACKGROUND

Sleep deprivation can affect an individual's health, safety and quality of life. For example, sleep deprivation can affect the individual's ability to drive safely and may increase their risk of other health problems. Signs and symptoms of sleep disorders include excessive daytime sleepiness, irregular breathing, increased movement during sleep, irregular sleep and wake cycle and difficulty falling asleep. Some common types of sleep disorders include: Insomnia, in which the individual has difficulty falling asleep or staying asleep throughout the night, sleep apnea, in which the individual experiences abnormal patterns in breathing while they are asleep, restless legs syndrome (RLS), in which the individual experiences an uncomfortable sensation and urge to move their legs while trying to fall asleep and Narcolepsy, a condition characterized by the individual falling asleep suddenly during the day. Doctors can usually treat most sleep disorders effectively once the disorders are correctly diagnosed. To properly diagnose a sleep disorder, doctor's must understand the patient's sleep patterns during the night. Self-reporting of sleep patterns by the patient are often inaccurate. For example, many people with sleep apnea are unaware of it, many people with Insomnia perceive less sleep than is measured and most normal people are unaware that they wake up 10-20 times per night.

To understand a patient's sleep patterns, doctors will typically perform objective sleep staging by monitoring Electroencephalographic (EEG) activity during sleep. An EEG is a test that detects electrical activity in the brain using electrodes attached to the scalp. A patient's brain cells communicate using electrical impulses and are active all the time even when the patient is asleep. Because sleeping with electrodes attached to the scalp can be cumbersome, other sensors for monitoring sleep patterns have been developed, such as wearable devices and in bed sensors. Wearable devices are typically worn on the wrist, legs or chest and include motion sensors (e.g., accelerometers) for tracking movements at those locations. In bed sensors are typically placed under a bed sheet and include sensors that can track breathing and heart rate by measuring tiny body movements that occur when a user breathes, or their heart beats. The sensor data can be input into a sleep staging application installed on a smartphone or other device. The sleep staging application computes various sleep metrics, such as total sleep/wake time and sleep efficiency, which can be used to quantify sleep to help users improve the amount of sleep they get, and to allow the sleep/wake tracking application to coach the users on how to get more sleep.

### SUMMARY

Embodiments are disclosed for sleep/wake tracking using machine learning.

In an embodiment, a method comprises: receiving, with at least one processor, sensor signals from a sensor, the sensor signals including at least motion signals and respiratory signals of a user; extracting, with the at least one processor, features from the sensor signals; predicting, with a machine learning classifier, that the user is asleep or awake based on the features; and computing, with the at least one processor, a sleep or wake metric based on whether the user is predicted to be asleep or awake.

In an embodiment, the features include at least respiratory rate variability, respiratory amplitude variability, movement periods and movement amplitudes.

In an embodiment, prior to extracting the features the features are transformed to approximate a specified distribution, and after the features are extracted the features are scaled to generalize the features.

In an embodiment, the method further comprises: estimating, with a temporal model, a path of sleep stage probabilities to improve the predicted sleep and wake probabilities based at least in part on transition probabilities.

In an embodiment, the temporal model includes a Viterbi path for providing the transition probabilities.

In an embodiment, the features include time-domain features and frequency-domain features.

In an embodiment, the frequency-domain features are computed by: low-pass filtering the sensor signals to remove noise; downsampling the filtered sensor signals; extracting, with a first window function, a first portion of the sensor signals; computing a mean of the first portion of the sensor signals; subtracting the mean from the first portion of the sensor signals; extracting, with a second window function, a second portion of the sensor signals; computing a frequency spectrum of the second portion of the sensor signals; and computing the frequency-domain features based at least in part on the frequency spectrum.

In an embodiment, the time-domain features are computed by: generating, with an activity detector, a stream of movement periods and amplitudes; extracting, with a window function, a portion of the movement periods and amplitudes; and computing, as the time-domain features, a fraction of time labeled as movement by the activity detector, a mean movement amplitude and maximum movement amplitude.

In an embodiment, the time-domain features are computed by: generating, with a breath detector, one or more streams of breath cycle lengths and breath cycle amplitudes; extracting, with one or more window functions, one or more portions of the one or more streams; and computing, as the time-domain features, at least one of a number of breaths, standard deviation, mean absolute deviation, root-mean-square (RMS) of successive differences, mean average deviation (MAD) of successive differences and range.

In an embodiment, the method of claim 1, wherein at least one feature is based on availability of sensor signals.

Other embodiments can include an apparatus, computing device and non-transitory, computer-readable storage medium.

Particular embodiments disclosed herein provide one or more of the following advantages. Machine learning is used to improve prediction of sleep/wake states that can be used by a sleep/wake tracking application to generate a variety of sleep metrics that can be used to quantify sleep to help users improve the amount of sleep they get, and to allow the sleep/wake tracking application to coach users on how to get more sleep.

The details of one or more implementations of the subject matter are set forth in the accompanying drawings and the description below. Other features, aspects and advantages of the subject matter will become apparent from the description, the drawings and the claims.

### BRIEF DESCRIPTION OF THE DRAWINGS

FIG. 1 illustrates sleep/wake tracking and sleep metrics, according to an embodiment.

FIG. 2 is a conceptual block diagram of a sleep/wake classification system, according to an embodiment.

FIG. 3 is a flow diagram of a sleep/wake tracking process performed by the system shown in FIG. 2, according to an embodiment.

FIG. 4 is a flow diagram of a feature extraction process, according to an embodiment.

FIG. 5 is a flow diagram of a classification process for predicting sleep/wake probabilities, according to an embodiment.

FIG. 6 is a flow diagram of a sleep/wake process, according to an embodiment.

FIG. 7 is a block diagram of a system architecture for implementing the sleep/wake features and processes described in reference to FIGS. 1-6, according to an embodiment.

FIG. 8 is a block diagram of a system architecture for a sleep/wake tracking device, according to an embodiment.

### DETAILED DESCRIPTION

### Example System

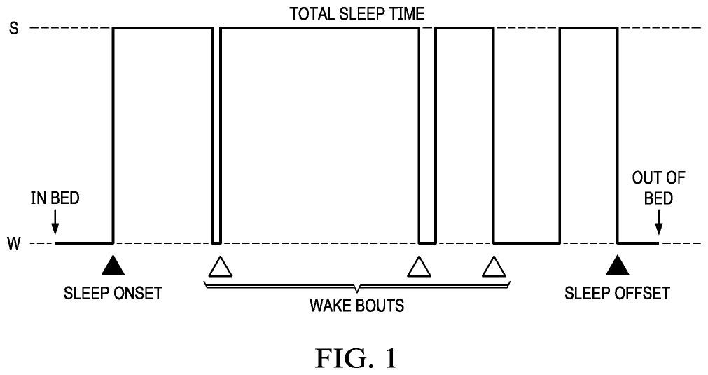

FIG. 1 illustrates sleep/wake tracking and sleep/wake metrics, according to an embodiment. An example sleep/wake time-series is shown, where “S” stands for “sleep time” and “W” stands for “wake time.” The total sleep time is interrupted by one or more “wake bouts” where the sleeper wakes up momentarily between Sleep Onset and Sleep Offset. Some example sleep metrics include but are not limited to: Total Sleep Time, Sleep Onset, Sleep Offset, In-Bed Time, Sleep Latency, Wake After Sleep Onset and Sleep Efficiency. Total Sleep Time is defined as the sum of all sleep times, Sleep Onset is defined as the start time of the first N minutes of sleep (e.g., N=4), Sleep Offset is defined as the end time of the last N minutes of sleep and Sleep Efficiency is defined as Total Sleep divided by Time in Bed. Other metrics may also be tracked by a sleep/wake tracking application.

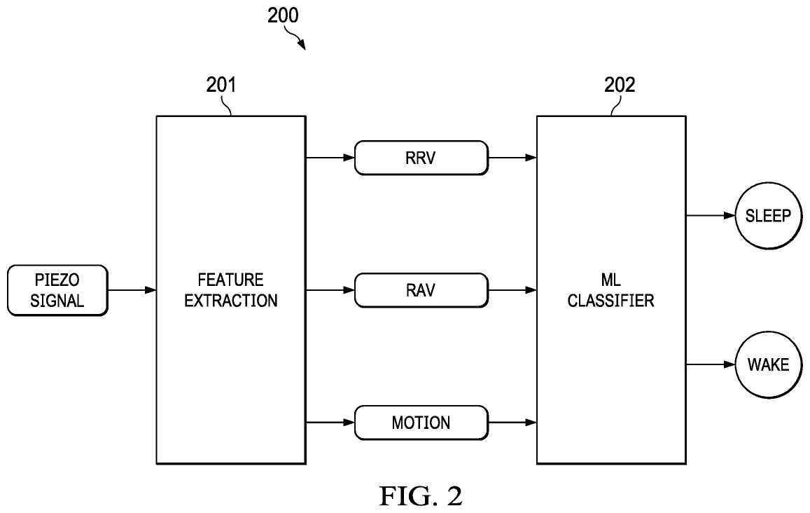

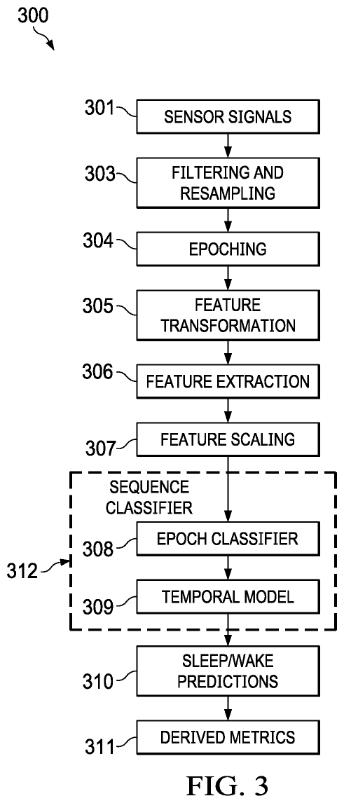

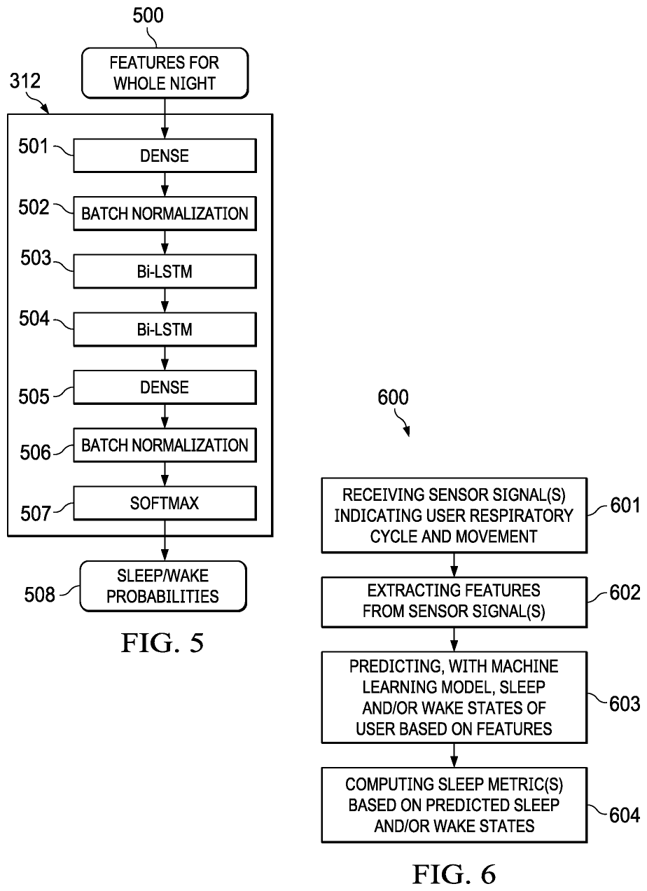

FIG. 2 is a conceptual block diagram of a sleep/wake tracking system 200, according to an embodiment. System 200 includes feature extractor 201 and machine learning (ML) classifier 202. Sensor signal(s) 202 from a sleep/wake tracking device (e.g., an in bed sensor) are input into feature extractor 201, which extracts multiple features, as described in reference to FIGS. 3-5. Some example features include but are not limited to: respiratory rate variability (RRV), respiratory amplitude variability (RAV) and motion. Other example features include but are not limited to heart rate (HR) and HR variability (HRV). These features are input into ML classifier 202 (e.g., as a feature vector), as described in reference to FIG. 5. ML classifier 202 predicts either a “sleep” or “wake” state based on the input features. In another embodiment, it is also possible for the ML classifier 202 to predict specific sleep stages such as REM, NREM1, NREM2, and NREM3 based on the input features.

In an embodiment, ML classifier 202 outputs a “sleep” or “wake” label and a probability of sleep or wake as a measure of confidence in the prediction (hereinafter, also referred to as “confidence score”). For example, if ML classifier 202 predicts an epoch of “sleep” with a 0.45 probability, that epoch would be classified as “wake” because its probability would be less than a specified threshold value (e.g., 0.55 in this example; sleep and wake probabilities add to one). Different probability thresholds can be used to tune the algorithm to more likely predict sleep or wake for any given epoch. From a full night of sleep/wake predictions, other derived metrics can be computed. For example, the Total Sleep Time metric can be computed by summing the sleep times between Sleep Onset and Sleep Offset. In another embodiment, ML classifier 202 generates a probability for each sleep stage (Wake, REM, NREM1, NREM2, NREM3), and the stage with the highest probability is chosen as the predicted stage.

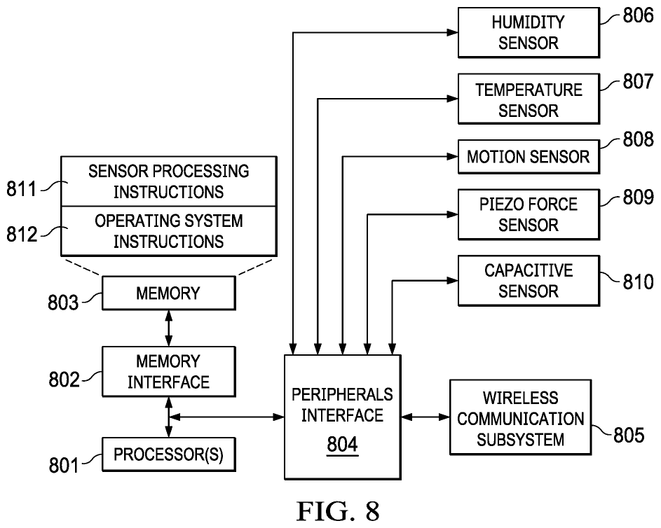

In an embodiment, the sleep/wake tracking device is a Beddit™ sleep tracker developed by Beddit™ Oy of Espoo Finland, which is an in bed sensor that connects to a smart phone or other companion device (e.g., a tablet computer, wearable device) using Bluetooth™ technology. The Beddit™ sleep tracker is a thin sensor strip that can be hidden under a bed sheet and includes a piezo force sensor and a capacitive touch sensor. Also included is a humidity sensor and a temperature sensor located in a dongle near the plug, as shown in FIG. 8. A sleep tracking application installed on the smartphone performs sleep analysis based on sensor signals received from the Beddit™ sleep tracker over the Bluetooth™ connection. The sleep analysis computes sleep metrics, including but not limited to: Sleep Onset, Sleep Offset, In-Bed Time, Total Sleep Time and Sleep Efficiency.

### Example Processes

FIG. 3 is a flow diagram of a sleep-wake tracking process 300 performed by the system 200 shown in FIG. 2, according to an embodiment. Process 300 includes filtering and resampling 303, epoching 304, feature transformation 305, feature extraction 306, feature scaling 307, epoch classifier 308 and temporal model 309. Hereinafter, epoch classifier 308 and temporal model 309 are collectively referred to as sequencer classifier 312.

Process 300 begins by filtering and resampling 303 sensor signals 301 (e.g., piezo signals) received from one or more sensors of a sleep/wake tracking device (e.g., an in bed sensor) to remove out of band noise. In an embodiment, the sensor signals include a piezo source signal that is sensitive to movements of the user's body due to breathing (from chest wall expansion), heart beats (from small movements related to the pumping of blood) and gross movement from shifting body positions or moving limbs. In an embodiment, the sensor signals representing heart rate (HR) are band passed filtered for a specified frequency band (e.g., between 0.5-40 Hz), the sensor signals representing breathing cycles is high pass filtered to remove low-frequency content (e.g., frequencies below 1 Hz) and the acceleration signal representing user body motion is high pass filtered to remove frequency content below a threshold (e.g., 10 Hz). In an embodiment, contributions to the sensor signals from movement, breathing and heart rate of a co-sleeper are detected and filtered from the sensor signals. In an embodiment, sensor signals are received from two or more sleep/wake tracking devices. In an embodiment, source signal separation techniques, such as independent component analysis or adaptive filtering, may be used to separate signals generated by two sleepers.

After filtering and resampling 303, epoching 304 is applied to the filtered signals. Epoching 304 generates windowed segments of the filtered peripheral signals for use by subsequent functions in process 300.

After epoching 304, feature transformation 305 is applied to the windowed segments. In an embodiment, feature transformation 305 transforms the features in the segments so the features are closer to a normal distribution. For example, a log transform can be applied to the segments to handle heavily skewed distributions.

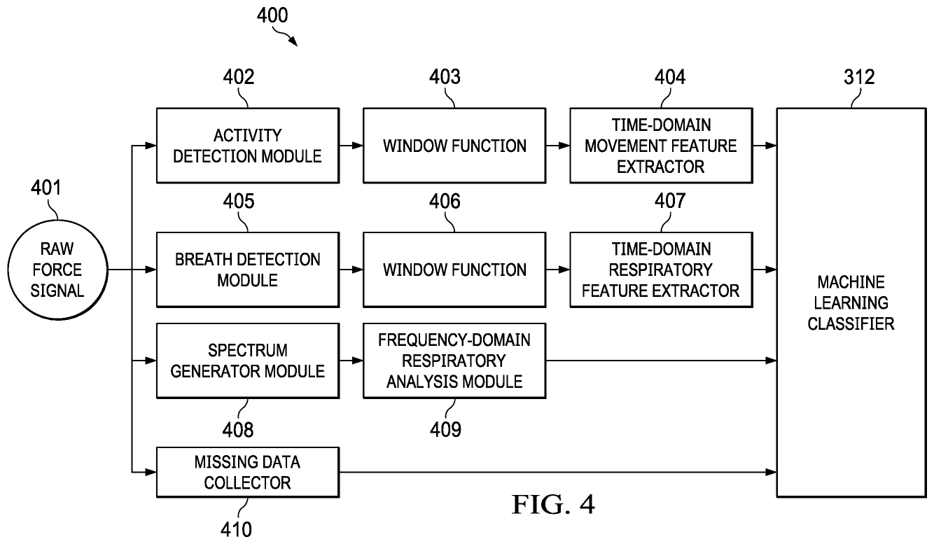

After feature transformation 305, feature extraction 306 is applied to the transformed segments. Feature extraction 306 extracts features that may be informative for sleep/wake classification. Some examples features useful for sleep/wake classification include but are not limited to: HR, HRV, RRV and Activity detection. Additional example features are described in reference to FIGS. 4 and 5.

After feature extraction 306, feature scaling 307 is applied to the extracted features to allow better generalization across subjects. For example, features in the 5^{th} percentile and 95^{th} percentile of a normal distribution can be scaled to 0 and 1, respectively, with zero mean and unit variance. In an embodiment, a tan h( ) function is applied to the features to reduce the effect of outliers and limit the range from −1 to 1.

After feature scaling 307, the scaled features are input into epoch classifier 308. Epoch classifier 308 predicts at each epoch a probability of the user being asleep or awake. In an embodiment, logistic regression is used to obtain sleep/wake probabilities based on the scaled features for each epoch.

The sleep stage probabilities output by epoch classifier 308 are input into temporal model 309, which improves the predictions by taking into account the temporal order of the epochs. In an embodiment, a deep neural network is used in place of epoch classifier 308 and temporal model 309, as described in reference to FIG. 5.

In an embodiment, a Viterbi path search, using learned sleep stage transition probabilities, is used to estimate the best sequence of sleep states (e.g. sleep/wake, or wake/REM/NREM1/NREM2/NREM3) over a period of time (e.g., over the course of night). The output of temporal model 309 are the final sleep stage predictions 310, which are used to compute sleep metrics 311. For example, Total Sleep Time can be computed by summing the epochs where a sleep state was predicted, i.e., summing the sleep times between wake bouts. The Viterbi path search utilizes the output probabilities from epoch classifier 308 as the state probabilities for each time-step, and finds the sequence of states that provides a maximum a posteriori probability. The state-transition probabilities (probability of transitioning between sleep stages) can be learned from a separate dataset.

FIG. 4 is a flow diagram of a feature extraction process 400, according to an embodiment. There are two main categories of features: time-domain features and frequency-domain features. In an embodiment, the frequency-domain features are obtained in real-time using streaming analysis, and the time-domain features are obtained in batch analysis from the results of the activity detection and breath detection streaming analysis at the end of a sleep period (e.g., at the end of the night). There are three processing paths for extracting features: time-domain movement detection path, time-domain respiratory cycle and frequency-domain respiratory analysis path. Each path receives as input a raw piezo force signal 401 from a sleep/wake tracking device (e.g., an in bed sensor). The raw piezo force signal captures tiny movements in the user's chest and abdomen due to breathing and heart beats. In an embodiment, the raw piezo force signal is generated by a strip of piezo film that produces a charge or voltage output when subjected to dynamic strain (change in its length). When the strip is mounted across a mattress in line with the user's chest/heart, the strip detects heart beats and changes in load or center of gravity due to the user's breathing. These signals, which are processed for an extended period of time (e.g., overnight), are indicative of duration, phase or quality of sleep. In other embodiments, other signals may also be processed if available (e.g. temperature, humidity).

In an embodiment, the time-domain movement detection path includes an activity detection module 402 that outputs a stream of movement states (“moving” or “not-moving”) and associated movement amplitudes whenever movement occurred. Window function 403 accumulates all movement periods and amplitudes within a window of N seconds (e.g., N=60). This window is shifted across the processing duration (e.g., whole night). Time-domain movement feature extractor 404 computes, from the window of movement periods and amplitudes, a mean movement amplitude, a maximum movement amplitude and a fraction of time the movement periods were labeled as movement by the classifier in the activity detection module 402. These time-domain movement features are then input into the sequencer classifier 312 shown in FIG. 3. Some examples of features output by the time-domain movement detection path include but are not limited to: High Activity Fraction, Activity Mean and Activity Max.

In an embodiment, the time-domain respiratory cycle extraction path includes a breath detection module 405 that outputs a stream of breath cycle lengths and a stream of breath cycle amplitudes. Window function 406 accumulates breath cycle lengths and amplitudes within an N second window (e.g., N=60), and time-domain respiratory feature extractor 407 extracts portions of the breath cycle lengths and breath cycle amplitudes for computing time-domain respiratory features. For example, from the respective windows of breath cycle lengths and breath cycle amplitudes, time-domain respiratory feature extractor 407 computes a standard deviation (SD), mean absolute deviation, root-mean-square (RMS) of successive differences, a mean average deviation (MAD) of successive differences and range can be computed as time-domain respiratory features to be input into the sequencer classifier 312 shown in FIG. 3. Some examples of time-domain respiratory features include but are not limited to: RRV SD, RAV SD, RRV MAD, RAV MAD, RRV RMSSD, RAV RMSSD, RRV MADSD, RAV MADSD, RRV Range, RAV Range and any other suitable features.

In an embodiment, the frequency-domain respiratory analysis path includes spectrum generator module 408, such as a Fast Fourier Transform (FFT), which outputs a frequency spectrum for the piezo signal. Prior to applying the frequency transformation, the raw piezo force signal 401 can be low-pass filtered to remove unwanted low-frequency noise. The signal is then downsampled and windowed. Additionally, the mean of the windowed data can be computed and subtracted from the windowed data and another window (e.g., Hann window) applied prior to the frequency transformation. In an embodiment, the frequency spectrum output of the FFT is folded (e.g., three harmonics), and the signal to noise (SNR) of peak power over power elsewhere in the spectrum is computed. The spectrum and SNR can be used by frequency-domain respiratory analysis module 409 computes a variety of frequency-domain respiratory features for input into the sequencer classifier 312 shown in FIG. 3. Some examples of frequency-domain respiratory features include but are not limited to: relative power for different bands, respiratory rate, respiratory SNR, full-width half-max (i.e., the width of a peak (in this example a peak in the FFT) computed at half of the maximum power), peak/mean power, Shannon entropy, kurtosis and any other suitable features.

In an embodiment, an additional feature is detected by missing data detector 410 based on the availability of data. This feature is “1” if, for any reason, there is not a full window of data available to compute all the features. This can occur, for example, when the user gets out of bed and the Bluetooth streaming disconnects and reconnects.

Note that the processing paths and features generated by the paths described above are examples, and more or fewer paths/features can be used/extracted to provide more or fewer features as input into sequence classifier 312 shown in FIG. 3. FIG. 5 is a flow diagram of deep neural network (DNN) 500 for predicting sleep/wake probabilities, according to an embodiment. In this example embodiment, DNN 500 utilizes recurrence (bi-directional), and therefore uses a full night of data to generate sleep/wake predictions (e.g., sleep/wake probabilities). In an embodiment, DNN 500 can replace just epoch classifier 308, just temporal model 309 shown in FIG. 3 or both. In this example embodiment, both the epoch classifier and temporal model are replaced with a DNN 500 that performs the functions of epoch classifier 308 and temporal model 309.

Classifier 500 receives feature vector 500 containing features for a whole night. The features can include, for example, all or some of the features described in reference to FIG. 4. The feature vector is input into dense layer 501, the output of which input into batch normalization layer 502, the output of which is input into bi-directional long short term memory (Bi-LSTM) network 503. Here, M is the number of measurement epochs. The output of Bi-LSTM network 503 is input into Bi-LSTM network 504. The output of Bi-LSTM network 504 is input into dense layer 505, the output of which is input into batch normalization layer 506, the output of which is input into softmax function 507. Softmax function 507 normalizes the output of batch normalization layer 506 to a probability distribution over predicted sleep stages.

Classifier 500 can be trained using back propagation techniques and annotated training data sets comprising data collected from sleep study participants. In other embodiments, other classification techniques can be used, such as a logistic regression, support vector machines (SVM), Extra Trees, Random Forests, Gradient Boosted Trees, Extreme Learning Machine (ELM), Perceptron and multi-layered convolutional neural networks.

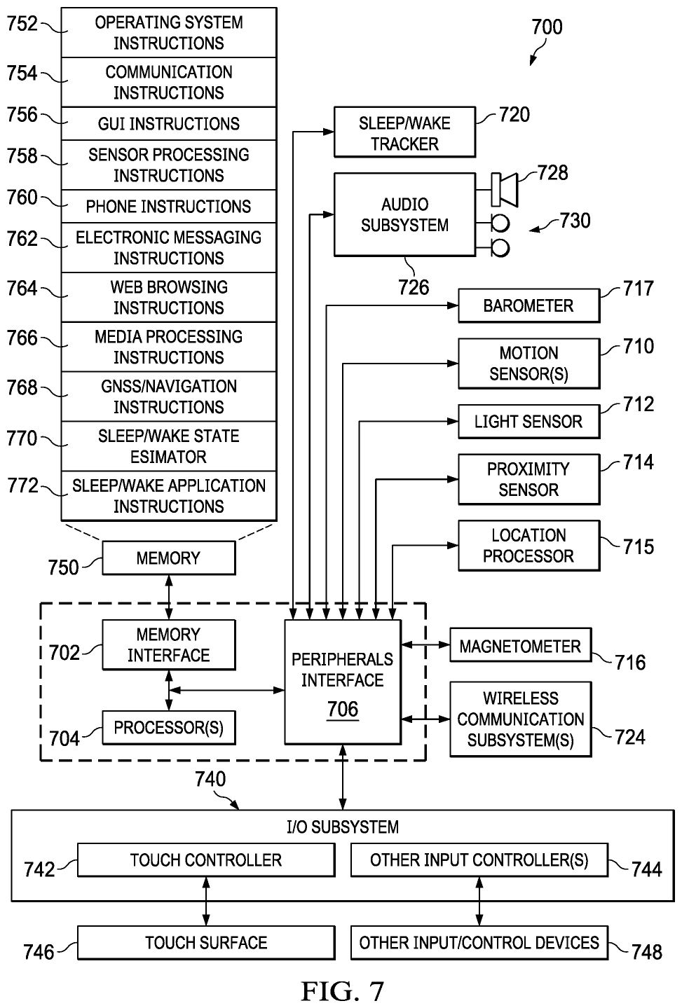

FIG. 6 is a flow diagram of process 600 of sleep tracking using machine learning, according to an embodiment. Process 600 can be implemented using the wearable device architecture 700 disclosed in reference to FIG. 7.

Process 600 includes the steps of receiving sensor signal(s) indicating a user's respiratory cycle and movement (601), extracting features from the sensor signal(s) (602), predicting, using machine learning model, sleep and/or wake states of the user based on the features (603), and computing sleep metric(s), based on the predicted sleep and/or wake states (604). Each of the preceding steps were described in detail in reference to FIGS. 2-5.

### Exemplary System Architectures

FIG. 7 illustrates example system architecture 700 implementing the features and operations described in reference to FIGS. 1-6. Architecture 700 can include memory interface 702, one or more hardware data processors, image processors and/or processors 704 and peripherals interface 706. Memory interface 702, one or more processors 704 and/or peripherals interface 706 can be separate components or can be integrated in one or more integrated circuits. System architecture 700 can be included in any suitable electronic device, including but not limited to: a smartphone, smartwatch, tablet computer, fitness band, laptop computer and the like.

Sensors, devices and subsystems can be coupled to peripherals interface 706 to provide multiple functionalities. For example, one or more motion sensors 710, light sensor 712 and proximity sensor 714 can be coupled to peripherals interface 706 to facilitate motion sensing (e.g., acceleration, rotation rates), lighting and proximity functions of the wearable device. Location processor 715 can be connected to peripherals interface 706 to provide geo-positioning. In some implementations, location processor 715 can be a GNSS receiver, such as the Global Positioning System (GPS) receiver. Electronic magnetometer 716 (e.g., an integrated circuit chip) can also be connected to peripherals interface 706 to provide data that can be used to determine the direction of magnetic North. Electronic magnetometer 716 can provide data to an electronic compass application. Motion sensor(s) 710 can include one or more accelerometers and/or gyros configured to determine change of speed and direction of movement. Barometer 717 can be configured to measure atmospheric pressure. Sleep/wake tracking subsystem 720 receives sensor signals from a sleep tracking device through a wired connection.

Communication functions can be facilitated through wireless communication subsystems 724, which can include radio frequency (RF) receivers and transmitters (or transceivers) and/or optical (e.g., infrared) receivers and transmitters. The specific design and implementation of the communication subsystem 724 can depend on the communication network(s) over which a mobile device is intended to operate. For example, architecture 700 can include communication subsystems 724 designed to operate over a GSM network, a GPRS network, an EDGE network, a Wi-Fi™ network and a Bluetooth™ network. In particular, the wireless communication subsystems 724 can include hosting protocols, such that the mobile device can be configured as a base station for other wireless devices. In an embodiment, wireless communication subsystems 724 includes a Bluetooth™ protocol stack for pairing with a sleep/wake tracking device, and for transferring sensor signals from the sleep-wake tracking device, such as the Beddit™ in bed sleep/wake tracking device.

Audio subsystem 726 can be coupled to a speaker 728 and a microphone 730 to facilitate voice-enabled functions, such as voice recognition, voice replication, digital recording and telephony functions. Audio subsystem 726 can be configured to receive voice commands from the user.

I/O subsystem 740 can include touch surface controller 742 and/or other input controller(s) 744. Touch surface controller 742 can be coupled to a touch surface 746. Touch surface 746 and touch surface controller 742 can, for example, detect contact and movement or break thereof using any of a plurality of touch sensitivity technologies, including but not limited to capacitive, resistive, infrared and surface acoustic wave technologies, as well as other proximity sensor arrays or other elements for determining one or more points of contact with touch surface 746. Touch surface 746 can include, for example, a touch screen or the digital crown of a smart watch. I/O subsystem 740 can include a haptic engine or device for providing haptic feedback (e.g., vibration) in response to commands from processor 704. In an embodiment, touch surface 746 can be a pressure-sensitive surface.

Other input controller(s) 744 can be coupled to other input/control devices 748, such as one or more buttons, rocker switches, thumb-wheel, infrared port and USB port. The one or more buttons (not shown) can include an up/down button for volume control of speaker 728 and/or microphone 730. Touch surface 746 or other controllers 744 (e.g., a button) can include, or be coupled to, fingerprint identification circuitry for use with a fingerprint authentication application to authenticate a user based on their fingerprint(s).

In one implementation, a pressing of the button for a first duration may disengage a lock of the touch surface 746; and a pressing of the button for a second duration that is longer than the first duration may turn power to the mobile device on or off. The user may be able to customize a functionality of one or more of the buttons. The touch surface 746 can, for example, also be used to implement virtual or soft buttons.

In some implementations, the mobile device can present recorded audio and/or video files, such as MP3, AAC and MPEG files. In some implementations, the mobile device can include the functionality of an MP3 player. Other input/output and control devices can also be used.

Memory interface 702 can be coupled to memory 750. Memory 750 can include high-speed random access memory and/or non-volatile memory, such as one or more magnetic disk storage devices, one or more optical storage devices and/or flash memory (e.g., NAND, NOR). Memory 750 can store operating system 752, such as the iOS operating system developed by Apple Inc. of Cupertino, Calif. Operating system 752 may include instructions for handling basic system services and for performing hardware dependent tasks. In some implementations, operating system 752 can include a kernel (e.g., UNIX kernel).

Memory 750 may also store communication instructions 754 to facilitate communicating with one or more additional devices, one or more computers and/or one or more servers, such as, for example, instructions for implementing a software stack for wired or wireless communications with other devices, such as a sleep/wake tracking device. Memory 750 may include graphical user interface instructions 756 to facilitate graphic user interface processing; sensor processing instructions 758 to facilitate sensor-related processing and functions; phone instructions 760 to facilitate phone-related processes and functions; electronic messaging instructions 762 to facilitate electronic-messaging related processes and functions; web browsing instructions 764 to facilitate web browsing-related processes and functions; media processing instructions 766 to facilitate media processing-related processes and functions; GNSS/Location instructions 768 to facilitate generic GNSS and location-related processes and instructions; and sleep/Wake estimator instructions 770 that implement the machine learning model and feature extraction processes described in reference to FIGS. 2-5. Memory 750 further includes a sleep/wake application instructions 772 for performing sleep analysis and computing sleep metrics used in the sleep analysis. For example, the sleep/wake application can use signals received from a sleep/wake tracking device 720 to perform sleep analysis, including but not limited to: detecting Random Eye Movement (REM) and non-REM sleep stages, computing sleep time, bedtime, time to fall asleep, time awake, time away from bed, wake-up time and sleep efficiency. Additionally, the application instructions 772 can provide instant visual and/or audio feedback regarding the results of a sleep analysis, display trend plots and provide sleep education through a display and/or audio.

Each of the above identified instructions and applications can correspond to a set of instructions for performing one or more functions described above. These instructions need not be implemented as separate software programs, procedures, or modules. Memory 750 can include additional instructions or fewer instructions. Furthermore, various functions of the mobile device may be implemented in hardware and/or in software, including in one or more signal processing and/or application specific integrated circuits.

FIG. 8 is a block diagram of a system architecture 800 for sleep/wake tracking device (e.g., an in bed sensor), according to an embodiment. System architecture 800 includes one or more processors 801, memory interface 802, memory 803, peripherals interface 804, wireless communication subsystem 805, humidity sensor 806, temperature sensor 807, motion sensor 808 and piezo force sensor 809 (piezo film) and capacitive touch sensor 810. Memory 803 includes sensor processing instructions 810 and operating system instructions 810. Memory 803 may also include one or more buffers for buffering data to be sent to another device (e.g., smartphone, wearable device (e.g., smartwatch)) using wireless communication subsystem 805, and protocol stacks implemented by wireless communication subsystem 805 (e.g., Bluetooth Protocol™ 4.2 stack, WIFI protocol stack). Sensor processing instructions 810 can compute various measurements based on sensor data, including but not limited to: average HR, highest HR, lowest HR and average breathing rate.

The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language (e.g., SWIFT, Objective-C, C#, Java), including compiled or interpreted languages, and it can be deployed in any form, including as a stand-alone program or as a module, component, subroutine, a browser-based web application, or other unit suitable for use in a computing environment.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

As described above, some aspects of the subject matter of this specification include gathering and use of data available from various sources to improve services a mobile device can provide to a user. The present disclosure contemplates that in some instances, this gathered data may identify a particular location or an address based on device usage. Such personal information data can include location-based data, addresses, subscriber account identifiers, or other identifying information.

The present disclosure further contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

In the case of advertisement delivery services, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

What is claimed is:

1. A method comprising:
receiving, with at least one processor, sensor signals from a sensor, the sensor signals including at least motion signals and respiratory signals of a user;
extracting, with the at least one processor, features from the sensor signals;
predicting, with a machine learning classifier, that the user is asleep or awake based on the features; and
computing, with the at least one processor, a sleep or wake metric based on whether the user is predicted to be asleep or awake.

2. The method of claim 1, wherein the features include at least respiratory rate variability, respiratory amplitude variability, movement periods and movement amplitudes.

3. The method of claim 1, wherein prior to extracting the features the features are transformed to approximate a specified distribution, and after the features are extracted the features are scaled to generalize the features.

4. The method of claim 1, further comprising:
estimating, with a temporal model, a path of sleep/wake probabilities to improve the predicted sleep and wake probabilities based at least in part on transition probabilities.

5. The method of claim 4, wherein the temporal model includes a Viterbi path for providing the transition probabilities.

6. The method of claim 1, wherein the features include time-domain features and frequency-domain features.

7. The method of claim 6, wherein the frequency-domain features are computed by:
low-pass filtering the sensor signals to remove noise;
downsampling the filtered sensor signals;
extracting, with a first window function, a first portion of the sensor signals;
computing a mean of the first portion of the sensor signals;
subtracting the mean from the first portion of the sensor signals;
extracting, with a second window function, a second portion of the sensor signals;
computing a frequency spectrum of the second portion of the sensor signals; and
computing the frequency-domain features based at least in part on the frequency spectrum.

8. The method of claim 6, wherein the time-domain features are computed by:
generating, with an activity detector, a stream of movement periods and amplitudes;
extracting, with a window function, a portion of the movement periods and amplitudes; and
computing, as the time-domain features, a fraction of time labeled as movement by the activity detector, a mean movement amplitude and maximum movement amplitude.

9. The method of claim 6, wherein the time-domain features are computed by:
generating, with a breath detector, one or more streams of breath cycle lengths and breath cycle amplitudes;
extracting, with one or more window functions, one or more portions of the one or more streams; and
computing, as the time-domain features, at least one of a number of breaths, standard deviation, mean absolute deviation, root-mean-square (RMS) of successive differences, mean average deviation (MAD) of successive differences and range.

10. The method of claim 1, wherein at least one feature is based on availability of sensor signals.

11. A system comprising:
at least one sleep staging device;
a host device comprising:
one or more processors;
memory storing instructions that when executed by the one or more processors, cause the one or more processors to perform operations comprising:
receiving, with at least one processor, sensor signals from the sleep/wake tracking device, the sensor signals including at least motion signals and respiratory signals of a user;
extracting features from the sensor signals;
predicting, with a machine learning classifier, that the user is asleep or awake based on the features; and
computing a sleep or wake metric based on whether the user is predicted to be asleep or awake.

12. The system of claim 11, wherein the sensor is a piezo force sensor.

13. The system of claim 11, wherein the machine learning classifier is a deep neural network that outputs sleep stage probabilities.

14. The system of claim 11, wherein prior to extracting the features the features are transformed to approximate a specified distribution, and after the features are extracted the features are scaled to generalize the features.

15. The system of claim 11, the operations further comprising:
estimating, with a temporal model, a path of sleep/wake probabilities to improve the predicted sleep and wake probabilities based at least in part on transition probabilities.

16. The system of claim 11, wherein the features are frequency-domain respiratory features computed by:
low-pass filtering the sensor signals to remove noise;
downsamping the filtered sensor signals;
extracting, with a first window function, a first portion of the sensor signals;
computing a mean of the first portion of the sensor signals;
subtracting the mean from the first portion of the sensor signals;
extracting, with a second window function, a second portion of the sensor signals;
computing a frequency spectrum of the second portion of the sensor signals; and
computing the frequency-domain features based at least in part on the frequency spectrum.

17. The system of claim 11, wherein the features are time-domain movement features computed by:
generating, with an activity detector, a stream of movement periods and amplitudes;
extracting, with a window function, a portion of the movement periods and amplitudes; and
computing, as the time-domain movement features, a fraction of time labeled as movement by the activity detector, a mean movement amplitude and maximum movement amplitude.

18. The system of claim 11, wherein the features are time-domain respiratory features computed by:
generating, with a breath detector, one or more streams of breath cycle lengths and breath cycle amplitudes;
extracting, with one or more window functions, one or more portions of the one or more streams; and
computing, as the time-domain features, at least one of a number of breaths, standard deviation, mean absolute deviation, root-mean-square (RMS) of successive differences, mean average deviation (MAD) of successive differences and range.

19. The system of claim 11, wherein at least one feature is based on availability of sensor signals.

20. The system of claim 11, wherein the features include at least respiratory rate variability, respiratory amplitude variability, movement periods and movement amplitudes.
